(19) United States
(12) Patent Application Publication
(10) Pub. No.: US 2007/0072293 A1
(43) Pub. Date: Mar. 29, 2007

(54) PROCESS FOR STABILIZATION OF BACTERIAL CELLS

(76) Inventor: Manoj Kole, Toronto (CA)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/535,233

(22) Filed: Sep. 26, 2006

Related U.S. Application Data
(60) Provisional application No. 60/719,989, filed on Sep. 26, 2005.

(51) Int. Cl. C12N 5/02; C12N 5/00; C12N 1/00

(52) U.S. Cl. 435/374; 435/243

(57) ABSTRACT

Biocompatible medium and non-ionic polymer surfactants such as tyloxapol may be used in a composition for preserving cells, such as bacterial cells. Cells may be preserved, for example, by suspending them in a biocompatible medium containing tyloxaopol and preserving the suspension. Examples of a preservation procedure are drying by foam formulation, glacification, dessication, spray drying, fluidized bed drying, drying in a vacuum, drying in a dry atmosphere, and drying at a high temperature in the range of 15-35° C.

### RELATED APPLICATION

This application claims benefit of U.S. provisional application Ser. No 60/719,989 filed Sep. 26, 2005 for “Stabilization of bacterial cells using tyloxapol.”

### BACKGROUND OF THE INVENTION

1. Field of the Invention

The invention generally relates to the stabilization of cells, particularly during preservation and storage procedures. In particular, the invention provides an improved method of stabilizing bacterial cells by adding a non-ionic liquid polymer surfactant, such as tyloxapol, to a medium in which the cell will be preserved. Furthermore the invention is enabling for certain stabilizing drying procedures by providing the ability to perform such procedures in a manner which allows the process to go forward and for reconstitution to occur

2. Backgrouind of the Invention

The long-term passage, preservation and storage of cells (e.g. bacterial cells) is of fundamental importance to research and biomedical endeavors. Several methods of preservation are known, such as freeze-drying (lyophilization), dessication, spray-drying, fluidized bed drying, drying in a vacuum, drying in a dry atmosphere, and drying by foam formation. U.S. Pat. No. 6,306,345 (Bronshtein et al.) discusses a foam-drying process for preserving bacteria by boiling a sample under vacuum to form a mechanically stable foam, dehydrating the foam to glassify the sample, and crushing the mechanically stable foam to form a powder U.S. Pat. N0. 6,509,146 (Bronshtein et al.) describes a similar procedure which also includes a secondary drying step, and cooling the dried material to a storage temperature that is lower than the glass transition temperature. U.S. Pat. No. 6,872,357 (Bronshtein et al.) describes preservation mnixtuires which comprise methylated monosaccharides and di-or oligosaccharides, and which can be used with a variety of preservation procedures.

Unfortunately, many bacteria do not survive these processes, and the low level of recovery of viable organisms is problematic. In particular, slow-growing mycobacterial strains tend to be highly susceptible to damage during preservation procedures, and the level of recovery of viable bacteria is typically very low. Furthermore, these procedures, to include lyophilization, foam drying and spray drying cannot be carried out for certain bacteria because of severe clumping and inability to reconstitute organisms initially in the process or later in attempts to reconstitute the dried material.

### SUMMARY OF THE INVENTION

The present invention provides improved methods and compositions for the stabilization of cells and for their preservation and storage. In a preferred embodiment of the invention, the cells are bacterial cells, particularly those that are difficult to grow and preserve (such as BCG and mycobacterium cells). The methods and compositions of the invention are based on the surprising discovery that the addition of a non-ionic polymer surfactant, such as tyloxapol (Triton 1339), to the medium used for culturing, preserving or storing mycobacteria results in high levels of viability, particularly after foam drying processes using one or more sugars (e.g., polysaccharides, disaccharides and monosaccharides).

### BRIEF DESCRIPTION OF THE DRAWINGS

FIG. 1. BCG stability pre- and post-foam drying. ♦=media containing methyl alpha-pyranoside/raffinose (MR); ▪=media containing sucrose/ raffinose (SR).

FIG. 2. Post-foam drying stability of BCG (Danish 1331) incubated (or “stored”) at 4° C., 25° C. and 37° C.

FIG. 3. Post-foam drying stability of BCG (Danish 1331) incubated (or “stored”) at 37° C.

FIG. 4. Post-foam drying stability of BCG (Danish 1331) incubated (or “stored”) at 25° C.

FIG. 5. Post-foam drying stability of BCG (Danish 1331) incubated (or “stored”) at 4° C.

FIG. 6. Chemical structure of tyloxapol.

FIG. 7. Foam-dry stability study of BCG Danish 1331 using 10% methyl alpha-glucopyranoside, 20% raffinose, 0.05% Tyloxapol formulation.

FIG. 8. Foam-dry stability study of BCG Danish 1331 using 20% Sucrose, 10% raffinose, 0.05% Tyloxapol formulation.

### DETAILED DESCRIPTION OF THE PREFERRED EMBODIMENTS OF THE INVENTION

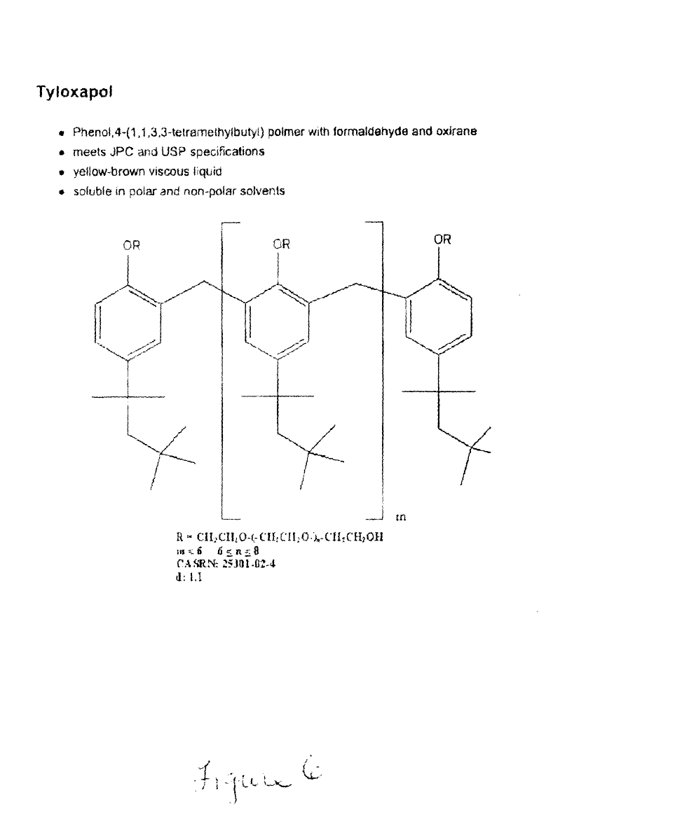

The present invention provides improved methods and compositions for the stabilization of cells in culture and during preservation and storage. The methods and compositions involve the addition of a non-ionic polymeric liquid surfactant, such as tyloxapol (Triton 1339), to the medium that is used for culturing, preserving or storing the cells. The chemical structure of tyloxapol, as available from Sigma and other sources, is shown in FIG. 6. Other non-ionic polymeric liquid surfactants may be used in the same manner as described herein below with reference to tyloxapol for exemplary purposes. The addition of tyloxapol permits dispersion of the cells for further processing (without tyloxapol, cell clumping occurs), and results in high levels of cell viability after preservation procedures, such as foam-drying, are performed.

Tyloxapol (FIG. 6) is a phenol,4-(1,1,3,3-tetramethylbutyl) polymer with formaldehyde and oxirane. Tyloxapol meets JPC and USP specifications; is a yellow-brown viscous liquid; and is soluble in polar and non-polar solvents. In the formula for Tyloxapol shown in FIG. 6, R=CH₂CH₂O—(—CH₂CH₂O—)ₙ—CH₂CH₂OH; m<6; 6≦n≦8 (CASRN: 25301-02-4; d: 1.1).

Those of skill in the art will recognize that many types of cells can be cultured or preserved using the methods of the present invention. Examples of such cells include but are not limited to bacterial cells, mammalian cells, plant cells, single-celled organisms, yeast, actinomycetes, and cells containing high amounts of fatty acids (high fatty acid content allows the cells to float). By “high fatty acid content” we mean that the cells contain at least about 50% fatty acids.

In one embodiment of the invention, the cells are bacterial cells, examples of which include but are not limited to corynebacteria species, mycobacterium species, Mycobacterium tuberculosis, Mycobacterium leprae, BCG, and recombinant forms thereof. In a preferred embodiment of the invention the cells are from various strains of Mycobacterium tuberculosis such as Danish (1331), Tice, Quebec, Pasteur, and Russian. Further, the method may also be applied to the culture and preservation of viruses.

The method of the invention results in the retention of a high level of cell viability during cell culture or during cell preservation procedures. Those of skill in the art will recognize that the method can be practiced with many preservation procedures, including but not limited to glacification, dessication, spray-drying, fluidized bed drying, drying in a vacuum, drying in a dry atmosphere, and drying by foam formation, and drying at high temperature (e.g. 14-35° C). Examples of such preservation procedures are given, for example, in issued U.S. Pat. No. 6,509, 146; 6,306,345; 6,537,666; and 6,872,357, the complete contents of which are hereby incorporated by reference. In addition, by “high level of cell viability” we mean that the cell culture, after undergoing a preservation procedure retains at least about 60%, preferably about 70%, more preferably about 80%, and most preferably about 90-100% or more of the viability that is measured in a comparable control culture of cells that have not undergone the preservation procedure. Those of skill in the art are familiar with the use of controls in experiments, and of the comparison of experimental to control results, and with determining the significance of those results.

Those of skill in the art will recognize that the type of media to which tyloxapol is added to culture and preserve the cells will vary from cell to cell, depending on the growth requirements of the cell. In general, the media must be “biocompatible” with the cells, by which we mean that the media must have a composition that is sufficient to sustain growth of the cell, as would be understood by one of skill in the art. Many such medias are known and can be used, examples of which include but are not limited to Middlebrook, Sultan, potato-glycerol (Lowenstein medium), and the like. The media may be either liquid or solid.

In the practice of the present invention, the non-ionic liquid polymer surfactant, such as tyloxapol, is added to a suitable biocompatible medium in order to culture and/or preserve the bacteria being grown in the medium. The amount added can vary (e.g., 0.01% to 5% being preferable). Depending on the circumstances, the non-ionic polymer surfactant (e.g., tyloxapol) may be added during any growth stage of the bacteria (e.g. during early, log or stationary phase). Alternatively, non-ionic polymer surfactant (e.g., tyloxapol) may be added to media in which the bacteria are re-suspended after being concentrated. In some cases, non-ionic polymer surfactant (e.g. tyloxapol) may be added only to the final media in which a bacteria is to be preserved and stored.

In general, the amount of tyloxapol added to the media will be in the range of about 0.01to about 5%, (weight/volume). In a preferred embodiment, the amount is about 0.05% (e.g., 0.5 g/L) to about 0.1% (wt/vol).

Those of skill in the art will recognize that other substances may also be added to media in order to promote the growth or stability of the bacteria being cultured or preserved. Examples of such substances include but are not limited to sugars, (e.g. glucose, sucrose, raffinose, oligosaccharides); sugar alcohols (such as mannitol, glycerol, sorbitol, etc.); antibiotics; vitamins; metal ions (e.g. Zn); substances to complement auxotrophy (e.g. amino acids); etc.

### EXAMPLES

During attempts to grow and concentrate mycobacterial cultures for preservation and long term storage, it was noted that it was very difficult to resuspend concentrated pellets of mycobacterium. Attempts at resuspension resulted in severe clumping and incomplete resuspension of the bacteria. Mechanical force is not an alternative because the use of mechanical force during resuspension disrupts the cells. Thus, it was not possible to obtain accurate OD readings, and working with the organisms (e.g. plating or sampling of the culture) was difficult and inaccurate. Surprisingly, we discovered that the addition of tyloxapol to the media greatly facilitated dispersion of the cells, resulting in a uniform resuspension of the mycobacterium. In order to ascertain the long-term effects of tyloxapol on bacteria, Mycobacteria grown and maintained in media containing tyloxapol were tested for growth and viability, particularly with respect to their survival after foam drying.

### Example 1

### Viability of Mycobacterium Maintained in Media Containing Tyloxapol Material and Methods

The organism employed in the study was Mycobacterium tuberculosis BCG Danish strain 1331.

For growing the mycobacteria, medium with the components listed in Table 1 was prepared with deionized water. The medium was distributed into flasks and sterilized at 120° C. for 30 minutes in an autoclave. The dextrose was sterilized separately and added to the medium sifter sterilization and before inoculation with mycobacteria. The pH of the initial medium was pH 6.9. Each flask was inoculated from a 10 ml sample of frozen seed stock with O.D.=3.3. After inoculation, the cultures were incubated in a gyrarotary incubator shaker at 120 rpm. Samples were taken every day and optical density (OD₅₄₀) was measured to observe the growth of the culture.

TABLE 1
Composition of Media

| Media composition: | (g/L) |
|---|---|
| Powdered Middlebrook 7H9 broth | 4.7 |
| Glycerol | 20.0 |
| Zinc sulfate | 0.03 |
| Magnesium sulphate | 0.2 |
| Sodium glutamate | 2.0 |
| Tyloxapol | 0.05 |
| Dextrose | 2.0 |

Sample Preparation

In preparation for foam-drying experiments, samples were taken from each flask and grown, and the colony forming units (CFU) were measured. The cultures were incubated overnight and growth and the CFU were also measured after the overnight incubation and before further processing.

The cultures were combined and divided into two portions and centrifuged, and the supernatant was discarded. Each of the two pellets was resuspended in one of two sugar formulation buffers (see below) such that the final volume of the resuspended material was 1/10th that of the culture volume at the time of centrifugation (i.e. a 1.2 liter total culture volume was used to make 60 ml of MR and 60 ml of SR formulation). A sample was taken from each of the sugar formulation for observing the initial CFU (i.e. CFU prior to foam drying).

Sugar formulations were made by adding one of the following to the media of Table 1:

1. MR: 15% MAG (Methyl-alpha-Glucopyranoside) +20% Raffinose +0.05% Tyloxapol
2. SR: 20% Sucrose +10% Raffinose +0.05% Tyloxapol
   Suitable vials were filled with 1 ml of the resuspended mycobacterial sugar formulations and lyophilized according to known procedures. Foam-dried samples were stored at −20° C.
   Stability study:

Foam-dried samples were taken directly from the −20° C. freezer and used for the initial post-preservation CFU analysis. On the same day, vials from each formulation were divided into four groups per formulation and stored at 37° C., 25° C., 4° C. and −20° C., so that 12 vials per formulation were stored at each temperature. The samples from the 37° C. group were plated for viability every week (3 vials per week for 4 weeks). The samples stored at 25° C. were sampled for viability monthly (3 vials per month for 4 months). The samples from 4° C. were sampled for ('FU (viability) every three months

Results:

The optical density of the formulated combined culture prior to lyophilization was OD=1.96

| | | |
|---|---|---|
| The CFU of the combined culture before form- | 6.7E7 | CFU/ml |
| ulation was: | | |
| The CFU after formulation (“formulated bulk”) | MR = 1.4E8 | CFU/ml |
| and before foam-drying | SR = 1.6E8 | CFU/ml |
| Bulk material after fill into vial before foam- | MR = 2.1E8 | CFU/ml |
| drying and storage at −20° C. | SR = 2.97E8 | CFU/ml |
| CFU for formulated sample to be used for base | MR = 2.1E8 | CFU/ml |
| line comparison: | SR = 1.49E8 | CFU/ml |

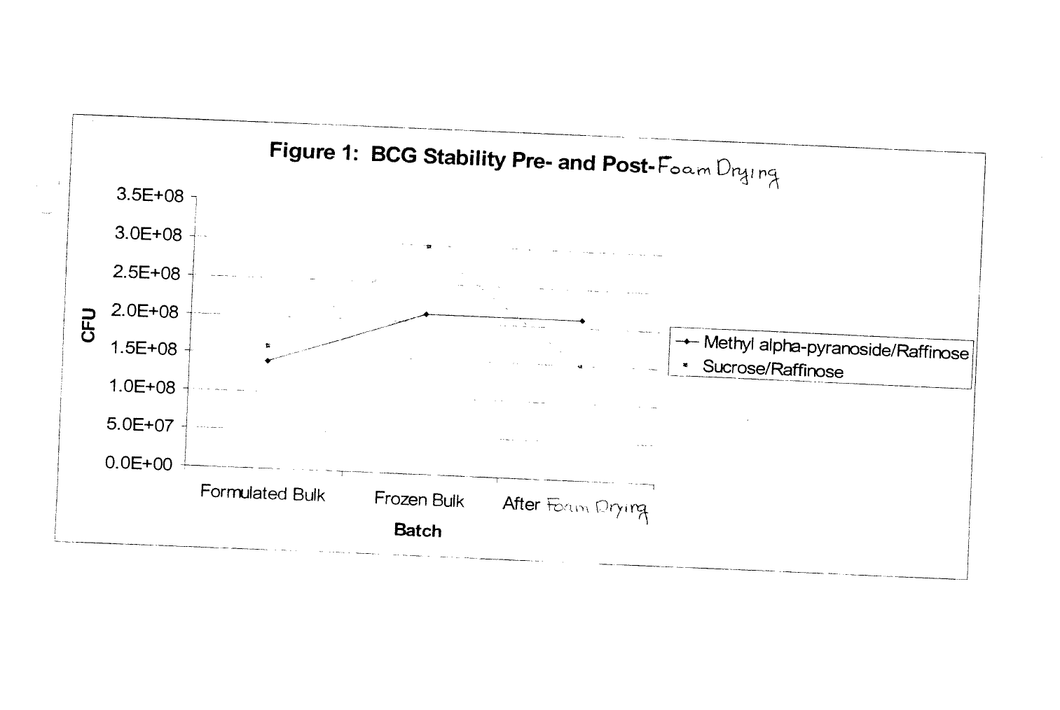

In FIG. 1, “formulated bulk” refers to the media containing bacteria resuspended in sugar formulation media and tyloxapol; “frozen bulk” refers to “formulated bulk” after freezing and thawing; and “after foam-drying” refers to bacterial samples that were foam-dried in media containing tyloxapol (the “formulated bulk” after a foam drying process). The data represents results obtained with the two different sugar formulations (SR and MR, described above). As can be seen, the mycobacteria foam-dried with both of the sugar formulations that contained tyloxapol displayed no major loss of viability as a result of foam-drying. In contrast, mycobacteria foam-dried by the standard procedure exhibited a 90% loss in viability, as measured by CFU.

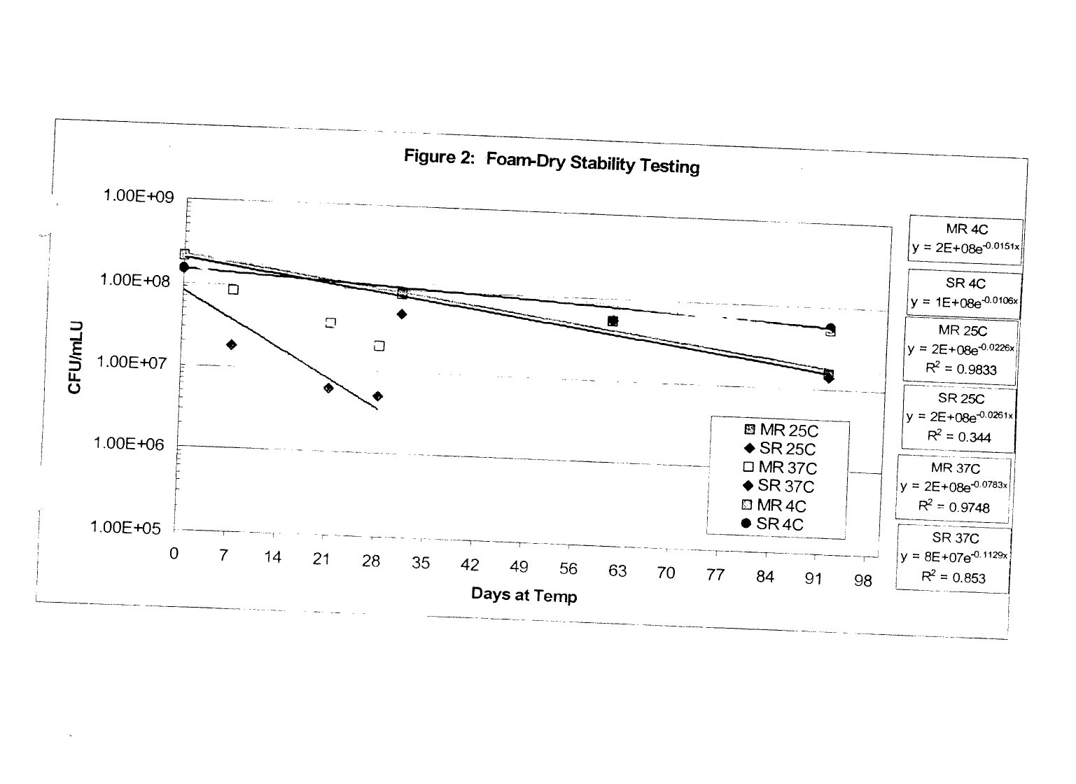

Studies of the long-term growth of bacteria from samples stored at three different temperatures (4° C., 25° C., and 37° C.) were carried out. The results are depicted graphically in FIG. 2. As can be seen, all cultures show a progressive loss of viability at each different temperature of incubation of the foam-dried material.

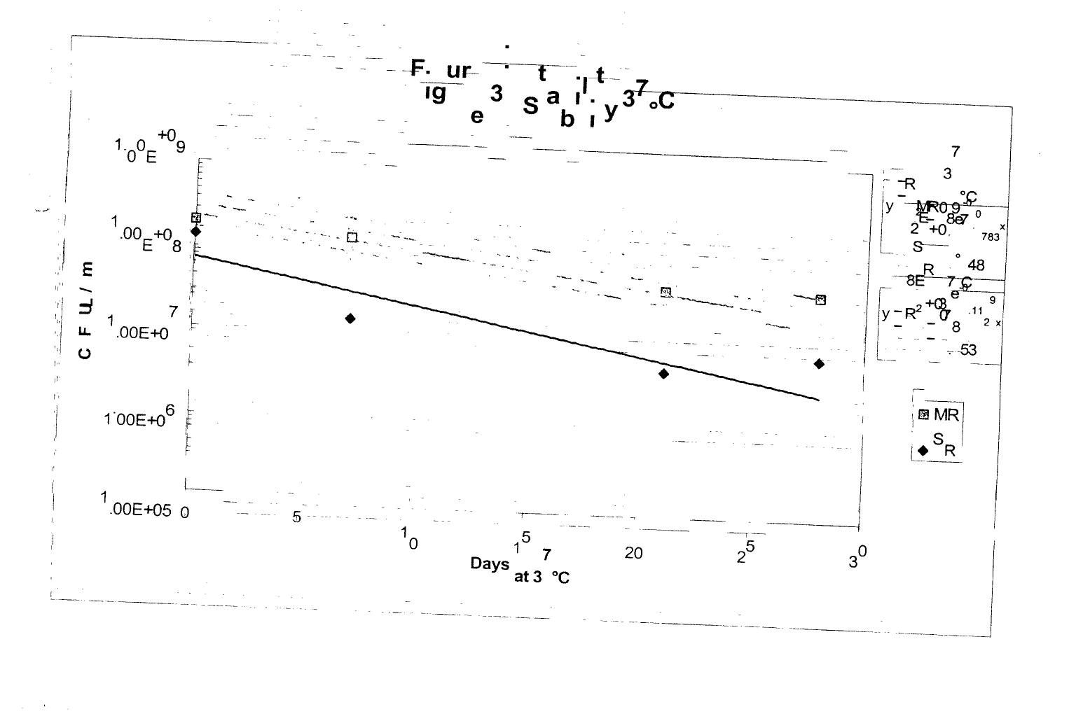

Data from individual temperatures is shown in FIGS. 3, 4 and 5. FIG. 3 shows that after one month at 37° C. the viability is lost by only 1.0 to 1.5 log. When compared to bacterial cultures that are to be foam-dried in media that does not contain tyloxapol, the present invention would provide superior results with respect to yielding dried formulations with viable cells. However, as a practical matter there is no ability to go forward with a comparison as formulations which lack tyloxapol are not able to be suspended (i.e., clumping and aggregation occurs). Thus, formulations of cell and media which lack tyloxapol, or an ingredient which has a similar affect on being able to suspend the cells, would not suitable for foam drying processes. Therefore, data in the Figures and Tables show that the addition of tyloxapol to the foam-drying media of bacterial cultures appears to result in increased viability at 37° C. These results are also presented in Table 2.

TABLE 2
Viability of Mycobacterium Cultured at 37° C. After Foam-Drying

Sugar Formulation**

| Time Point (Week)* | MR*** | SR*** |
|---|---|---|
| 0 | 2.10E+08 | 1.49E+08 |
| 1 | 8.30E+07 | 1.8E+07 |
| 2 | N/A | N/A |
| 3 | 3.70E+07 | 5.90E+06 |
| 4 | 3.40E+07 | 5.00E+06 |

*Samples were taken once a week for four weeks (except for week 2).
**All data represents CFU/ml.

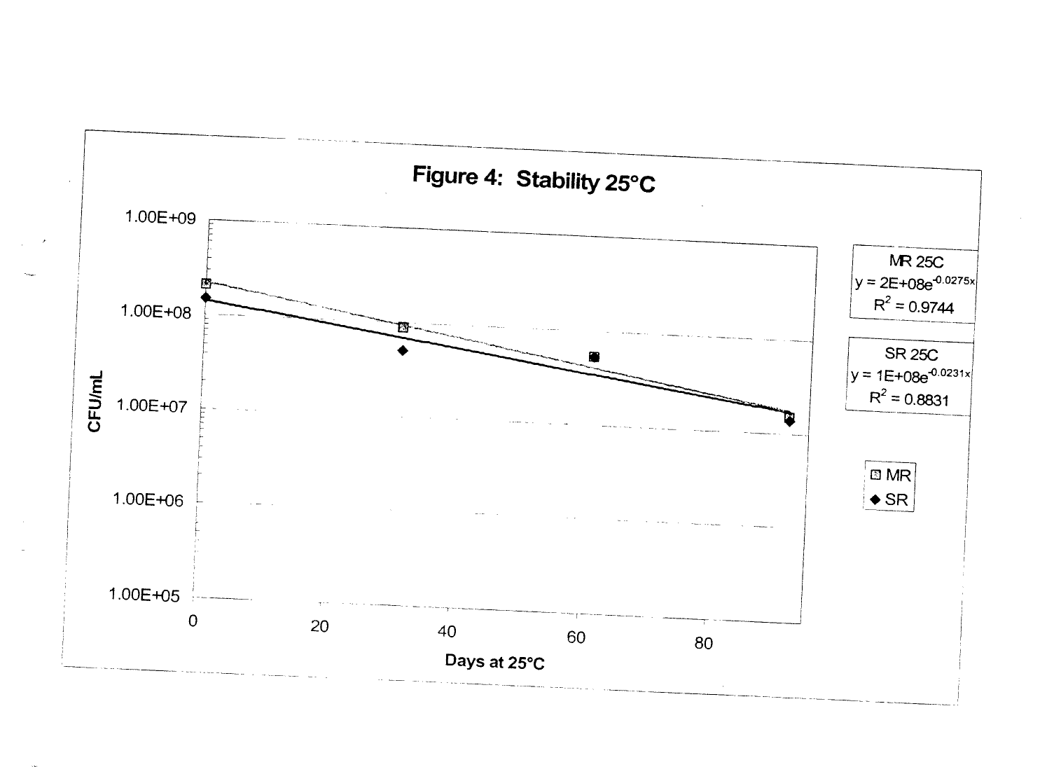

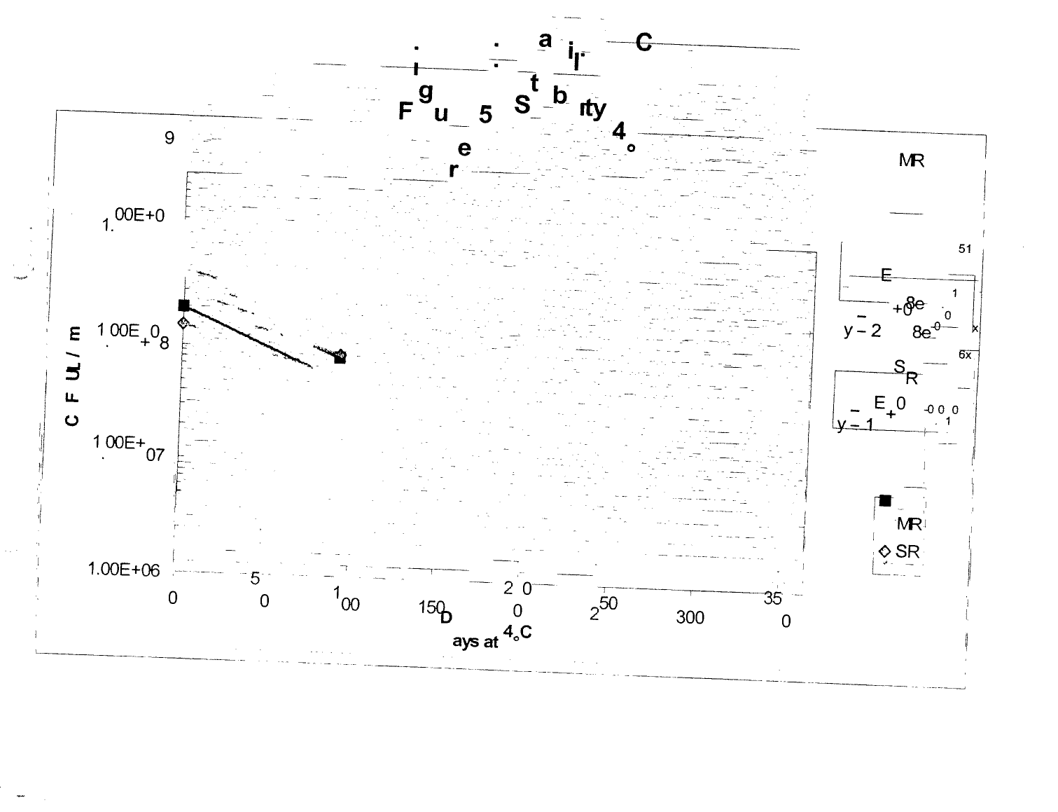

*** Formulations as described above: MR: 15% MAG (Methyl-alpha-Glucopyranoside) +20% Raffinose +0.05% Tyloxapol ; SR: 20% Sucrose +10% Raffinose +0.05% Tyloxapol
The results obtained after 90 days for cultures stored at 25° C. are depicted in FIG. 4. As can be seen, viability is lost by approximately 1.0 log . At 4° C. (FIG. 5) viability is lost by 0.5 logs after 90 days of incubation.
Thus, at all incubation temperatures there is loss of viability, as is typical of most biological materials such as bacteria. However, the viability at 37° C. was improved by the addition of tyloxapol to the preservation media.
Conclusion:

This example demonstrates that the viability of the bacterial cells in media containing tyloxapol is 100% after foam-drying. Further, the addition of tyloxapol facilitates the dispersion of bacterial cells, e.g. after centrifugation. Similar results may be expected with other non-ionic liquid polymer surfactants.

### Example 2

### Foam-dry Stability Studies

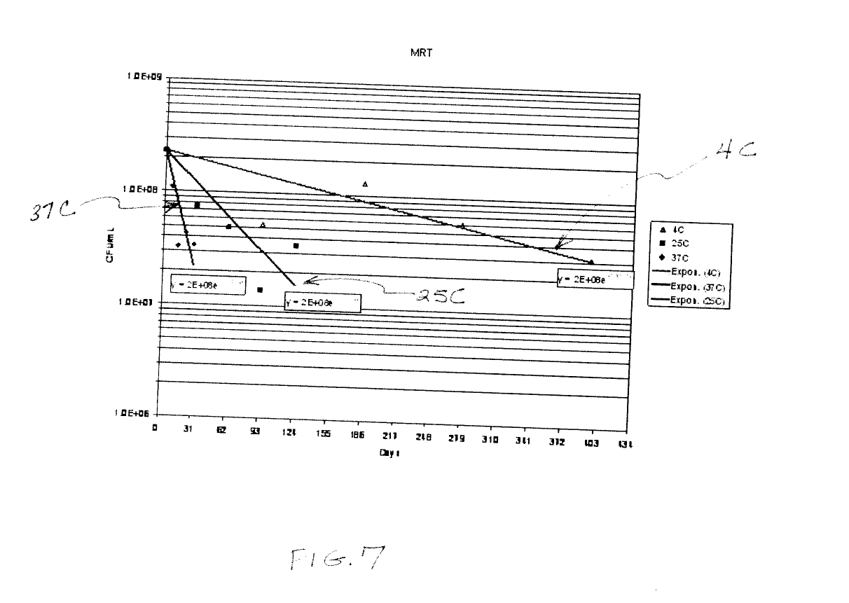

Experimentation was conducted in which foam-dry stability was studied for BCG Danish 1331 using 10% methyl alpha-glucopyranoside, 20% raffinose, 0.05% Tyloxapol formulation (FIG. 7). Stability was measured for each specimen using an exponential least-squares fit for CFU vs. Time. FIG. 7 is a composite of three temperatures 4° C., 25° C., and 37° C., the later study temperature has additional test data previously described in FIG. 3.

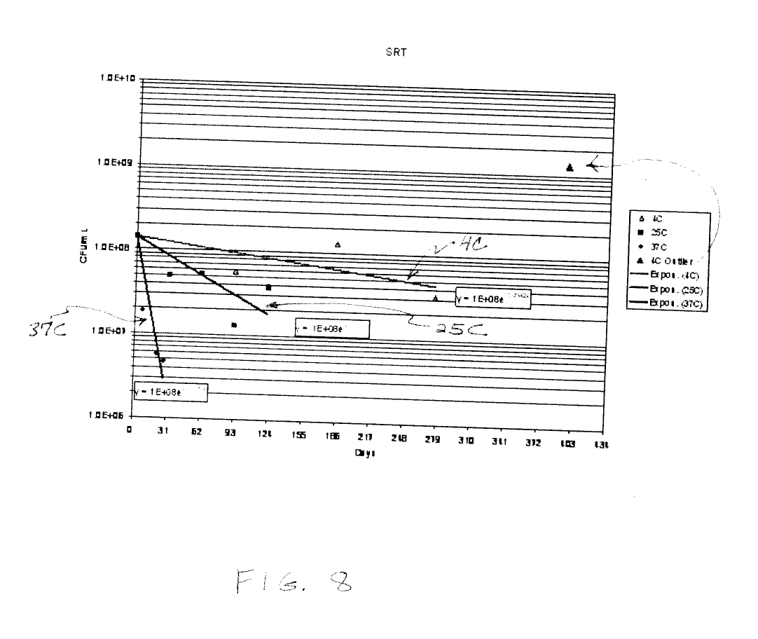

Experimentation was conducted in which foam-dry stability was studied for BCG Danish 1331 using 20% Sucrose, 10% raffinose, 0.05% Tyloxapol formulation (FIG. 8). Stability was measured for each specimen using an exponential least-squares fit for CFU v. Time. FIG. 8 is a composite of three temperatures 4° C., 25° C., and 37° C., the later temperature has additional test data previously described in FIG. 3.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

I claim:

1. A composition for preserving cells, comprising
biocompatible medium and
a non-ionic polymer surfactant.

2. The composition of claim 1, wherein said cells are bacterial cells.

3. The composition of claim 1 wherein said non-ionic polymer surfactant is tyloxapol.

4. The composition of claim 1 further comprising one or more monosaccharides, disaccharides, or polysaccharides, and wherein said biocompatible medium, said non-ionic polymer surfactant and said one or more monosaccharides, disaccharides, or polysaccharides are present in a dried foam.

5. A method for preserving cells, comprising the steps of
forming a suspension of said cells in a biocompatible medium containing a non-ionic polymer surfactant,
and
preserving said suspension by a procedure selected from the group consisting of drying by foam formulation, glacification, dessication, spray drying, fluidized bed drying, drying in a vacuum, drying in a dry atmosphere, and drying at a high temperature in the range of 15-35° C.

6. The method of claim 5 wherein said cells are bacterial cells.

7. The method of claim 5 wherein said preserving step is performed by foam formulation.

8. The method of claim 7 wherein said non-ionic polymer surfactant is tyloxapol.

9. The method of claim 7 wherein said foam formulation used in said preserving step includes one or more monosaccharides, disaccharides, or polysaccharides.

10. A mycobacterium composition, comprising mycobacteria preserved in a viable state in a dried foam comprising biocompatible media, a nonionic polymer surfactant, and one or more monosaccharides, disaccharides, or polysaccharides.

11. The mycobacterium composition of claim 10 wherein said nonionic polymer surfactant is tyloxapol.

12. The mycobacterium composition of claim 11 wherein said dried foam is pulverized or crushed.
